(19) United States
(12) Patent Application Publication
(10) Pub. No.: US 2002/0086048 A1
(43) Pub. Date: Jul. 4, 2002

(54) Design and application method for enhanced oral assimilation through the oral mucosa and/or intestinal membranes, of liquids containing nutrients, nutraceuticals or drugs, which are encapsulated in natural and/or synthetic liposomal vesicles

(76) Inventors: Arnold C. Takemoto, Scottsdale, AZ (US); Nikolaus J. Smeh, Stafford, VA (US)

Correspondence Address: Mr. Nikolaus J. Smeh, 28 Hulvey Drive, Stafford, VA, 22554, US

(21) Appl. No.: 09/750,678

(22) Filed: Jan. 2, 2001

(51) Int. Cl. A61K 31/375; A61K 9/127; A01N 37/12; A61K 31/195

(52) U.S. Cl. 424/450; 514/474; 514/566

(57) ABSTRACT

Method of oral administration of nutrients, nutraceuticals, and drugs to humans and animals orally by encapsulating these substances into vesicles formed by polar molecules, like phospholipids, for enhanced transport through oral mucosa or intestinal membranes into the circulatory system.

This invention related to and has for an object an unusual method of enclosing nutrients, nutraceuticals, and drugs into vesicles formed by polar molecules. These polar molecules are the same as nature uses to form the membranes of every living cell. They are synthesized in plants and animals and are called phospholipids. Under special circumstances, using electric forces, pressure and impact, these molecules form spheroidal shaped cells which enclose the liquid in which they are formed. The size of these cells vary but are of the order of nanometers to microns and can therefore easily penetrate oral and intestinal membranes.

Many substances, like nutrients, nutraceuticals and pharmaceuticals, are either not well tolerated by the human or animal digestive system or may be destroyed by the digestive system before being able to pass through the intestinal membranes to perform their intended functions in the body. An example would be the oral administration of the chelation agent disodium ethylenediaminetetraacetic acid (EDTA) for the purpose of detoxification of the human body. Even very small amounts of oral disodium EDTA (500 milligram) cause a subject to suffer instant diarrhea while only less than 5% of the oral administered dose crosses the membrane barrier into the circulatory system. When an aqueous disodium EDTA solution is encapsulated in liposomes, nearly 100% of the solution passes the intestinal membrane into the circulatory system with little or no side effects i.e. diarrhea.

1 What we claim as our invention is any natural and synthetic liposome encapsulated or emulsified nutrient, nutraceutical, or drug intended for enhanced oral assimilation specifically for: EDTA, vitamin C, and Insulin, for the purpose of passing these substances into the circulatory system through the oral mucosa or intestinal membranes:

2 What we claim as our invention are natural and synthetic Liposome encapsulated liquids containing vitamin C derived from Corn, Beet, and Tapioca/Cassava, orther sources intended for enhanced oral assimilation for the purpose of passing these substances into the circulatory system through the oral mucosa or intestinal membranes.

3 What we claim as our invention are natural and synthetic Liposome encapsulated liquids containing Disodium EDTA intended for enhanced oral assimilation for the purpose of passing these substances into the circulatory system through the oral mucosa or intestinal membranes.

4 What we claim as our invention are natural and synthetic Liposome encapsulated liquids containing Insulin intended for enhanced oral assimilation for the purpose of passing these substances into the circulatory system through the oral mucosa or intestinal membranes.
